(19) United States
(12) Patent Application Publication
(10) Pub. No.: US 2010/0297616 A1
(43) Pub. Date: Nov. 25, 2010

(54) SITU HYBRIDISATION METHOD

(75) Inventors: Mahesh Arjandas Choolani, Singapore (SG); Sze Yee Sherry Ho, Singapore (SG); Arijit Biswas, Singapore (SG); Khalil Razvi BM Jabarullah Khan, Loyang Valley (SG)

Correspondence Address: CLARK & ELBING LLP, 101 FEDERAL STREET, BOSTON, MA, 02110, US

(73) Assignee: NATIONAL UNIVERSITY OF SINGAPORE, Singapore (SG)

(21) Appl. No.: 12/294,766

(22) PCT Filed: Mar. 27, 2007
(86) PCT No.: PCT/SG2007/000082

§ 371 Date: Sep. 26, 2008

(30) Foreign Application Priority Data
Mar. 27, 2006 (SG) 200601942-6

(51) Int. Cl. C12Q 1/68; G01N 33/68

(52) U.S. Cl. 435/6; 435/7.1

(57) ABSTRACT

There is provided an improved method of in situ hybridisation. In particular, a method of in situ hybridisation of biopolymer(s) in a sample of cells, with the proviso that the digestion of polypeptides is not performed. The method may be applicable to detection of different types of biopolymers, including detection of nucleic acids and polypeptides in the same sample. In particular, the method is for the detection of chromosomal abnormalities in uncultured amniotic cells.

### FIELD OF THE INVENTION

The present invention relates to an improved method of in situ hybridisation for detecting the presence and/or location of biopolymers in cells or histology samples.

### BACKGROUND OF THE INVENTION

The in situ hybridisation (ISH) is a commonly used technique in the field of biomedical science. ISH is used to reveal the presence of biopolymers of interest as well as show their location in the cell or tissue. Cells or tissue of interest are mounted onto a solid surface such as a glass slide and one or more probes complementary to the target biopolymer of interest are allowed to hybridize or bind to biopolymer. Unbound probe is then removed by washing. The presence or location of the bound probe that remains is then detected. Improvements to the ISH technique have been described in U.S. Pat. No. 5,871,932 and U.S. Pat. No. 6,022,689.

One variation of ISH is fluorescent ISH (or FISH) that is gaining in popularity, for example, for the detection of chromosomal abnormalities in amniotic cells drawn from expectant women.

Fluorescence in situ hybridisation (FISH) relies upon visual counting of fluorescence signals within target fetal cells rather than comparing fetal with parental genotypes. In all cases where hybridisation is successful, the results are informative, and the technique can also be used for other suspected chromosomal rearrangements such as DiGeorge syndrome (DGS) (Jouannic et al, 2003). Abnormal results may be important in clinical decision-making, but normal results allay much of the anxiety associated with the longer wait for the full karyotype (Marteau et al, 1992).

Since the first clinical programme using FISH on uncultured amniotic fluid cells (Ward et al, 1993), several centres worldwide have begun offering this service routinely to patients undergoing midtrimester amniocentesis (Witters et al, 2002). The typical reporting time of FISH protocol of Vysis (Downers Grove, Ill., USA) is 48 hours (http://www.vysis.com).

Ideally, specific indication of the normality of the fetus should be available on the same day as the diagnostic invasive procedure. This would be possible if results of the analysis of uncultured amniotic fluid cells after invasive testing' were ready in less than the two days required by the methods of the prior art.

### SUMMARY OF THE INVENTION

The present invention addresses the problems above, and provides an improved and efficient method of in situ hybridisation of biopolymer(s).

According to a first aspect, the present invention provides a method for in situ hybridisation of biopolymer(s), for example nucleic acid(s) and/or polypeptide(s), in at least one isolated cell with the proviso that the method does not comprise digestion of polypeptide(s). In particular, there is provided a method for in situ hybridisation of nucleic acid(s) in at least one isolated cell with the proviso that the method does not comprise digestion of polypeptide(s).

More in particular, there is provided a method for in situ hybridisation of nucleic acid(s) and/or polypeptide(s) in at least one isolated cell comprising the steps of: (a) providing at least one isolated cell; (b) contacting at least one cell with at least one hypotonic solution; (c) treating the at least one cell with at least one fixative; (d) providing at least one molecule capable of binding to at least one nucleic acid and/or polypeptide, and allowing binding of the at least one molecule to the nucleic acid(s) and/or polypeptide(s) for a time period; (e) removing any unbound molecule(s); and (f) detecting any bound molecule(s), with the proviso that method does not comprise digestion of polypeptides.

The method may be used for detection of different biopolymers such as nucleic acids and polypeptides in the same sample of cells by use of at least one molecule which binds to at least one nucleic acid and/or at least one molecule which binds to at least one polypeptide. The at least one (first) molecule may be a molecule complementary to the at least one nucleic acid and/or the at least one (second) molecule may be a molecule complementary to the at least one polypeptide.

Further, the molecule(s) capable of binding to at least one nucleic acid and/or polypeptide may be labeled with at least one label selected from the group consisting of fluorescer, chemilumimescer, enzyme label and radiolabel.

In particular, denaturing of biopolymer(s) may be performed at a temperature range from 50° C. to 70° C. For example, at a temperature range from 55° C. to 65° C., in particular at a temperature of approximately 60° C. In the method of the invention, the sample of cells may or may not be dehydrated. In particular, the sample of cells is not dehydrated.

In the method of the invention, the binding time period of step (d) is less than 60 minutes; in particular, the binding time period is less than 45 minutes; more particularly, the binding time period is less than 20 minutes.

In the method, step (d) comprises at least one first temperature exposure at a temperature range from 70° C. to 90° C. for 60 sec to 120 sec. In particular, the first temperature exposure is at a temperature range from 75° C. to 85° C. for 80 sec to 100 sec. In particular, the first temperature exposure is at a temperature of approximately 80° C. for 90 sec. Step (d) further comprises at least one subsequent temperature exposure at a temperature range from 37° C. to 47° C. In particular, the subsequent temperature exposure is performed at a temperature range from 40° C. to 45° C. In particular, the subsequent temperature exposure is of approximately 42° C.

In the method of the invention, the cells are eukaryotic, for example mammalian cells. The mammalian cells may be human cells or non-human cells. More particularly, the cells may be human amniotic cells. The method may further comprise a step of diagnosis. The step of diagnosis may comprise determining the presence or absence of chromosomal abnormalities in the cell(s) of a mammal. In particular, there is provided a method for determining the normality or abnormality of a fetus.

According to one aspect of the invention, the method may be used for detecting chromosomal abnormalities in amniotic cells. In particular, the amniotic cells may be obtained from pregnant mammals. Accordingly, there is provided a method for detecting chromosomal abnormalities in amniotic cells comprising the steps of:
(a) providing at least one cell isolated from amniotic fluid;
(b) contacting the at least one cell with at least one hypotonic solution;
(c) treating the at least one cell with at least one fixative;
(d) providing at least one molecule capable of binding to at least one nucleic acid and/or polypeptide, and allowing binding of the at least one molecule to the nucleic acid(s) and/or polypeptide(s) for a time period;
(e) removing any unbound molecule(s);
(f) detecting any bound molecule(s); and
(g) detecting the presence or absence of chromosomal abnormalities, with the proviso that the method does not comprise digestion of polypeptides.

In the method of the invention, the method may also be used for, detecting presence of at least one polypeptide in a sample of cells. In particular, in the method according to the invention, the presence of chromosomal abnormality(ies) correlates to the abnormality of a fetus.

According to another aspect, the present invention provides a kit for assaying the presence of a suspect nucleic acid and/or polypeptide in a cell sample comprising at least one hybridisation buffer salt, a denaturing agent, a hybridising agent, a salt for a hypotonic solution, a fixative, at least one molecule capable of being detected and optionally information pertaining to the kit. In particular, the kit according to the invention does not include any protease. More in particular, there is provided a kit wherein the kit is for detecting chromosomal abnormalities is amniotic cell(s). More in particular, the kit according to the invention is for diagnosis the normality or abnormality of a fetus. The kit may further comprise instructions and/or illustrations for use.

### BRIEF DESCRIPTION OF THE FIGURES

FIG. 1 is a flowchart showing the methods of the prior art and that according to one particular embodiment (see Example) of the present invention. FIG. 1(A) shows the FISH method of the closest prior art from Vysis.

FIG. 1(B) shows a protocol described in the Example of a method according to the invention. However, the present invention is not limited to the specific reagents, amounts and times indicated in the protocol shown in FIG. 1(B).

For FIGS. 2 to 7, the following colours have been used: centromeric enumeration probe (CEP), CEP X (red); CEP Y (green); CEP 18 (aqua); Locus-Specific (LSI) Probe, LSI 21 (yellow); LSI 13 (purple) and are labelled accordingly. Colours are enhanced for ready recognition.

FIG. 2 shows cells at interphase stage.

FIG. 2A shows CEP X and CEP Y are hybridised in nuclei of uncultured male amniocytes

FIG. 2B shows CEP X hybridised in nuclei of uncultured female amniocytes.

FIG. 2C shows a normal diploid in chromosome 21 of uncultured female amniocytes.

FIGS. 3 and 4 show different trisomy conditions.

FIG. 3A shows CEP 18 is hybridised in nucleus of an uncultured amniocyte with three copies of chromosome 18 (Edward's Syndrome).

FIG. 3B shows LSI21 is hybridised in nucleus of an uncultured amniocyte showing three copies of chromosome 21 (Down's Syndrome).

FIG. 4A shows LSI 13 is hybridised in nucleus of an uncultured amniocyte with three copies of chromosome 13.

FIG. 4B shows CEP X and CEP Y is hybridised in nucleus of an uncultured amniocyte with two copies of chromosome X and one copy of chromosome Y (47 XXY, Klinefelter syndrome)

FIGS. 5 and 6 shows mononuclear cells from fetal blood.

FIG. 5A shows CEP X and CEP Y hybridised in nuclei of mononuclear cells from male fetal blood.

FIG. 5B shows normal diploid in chromosome 21 and chromosome 13 of mononuclear cells from fetal blood.

FIG. 6A shows CEP 18 is hybridised in nucleus of mononuclear cell from male fetal blood with three copies of chromosome 18 (Edward's Syndrome).

FIG. 6B shows LSI21 is hybridised in nucleus of mononuclear cell showing three copies of chromosome 21 (Down's Syndrome).

FIG. 6C shows CEP X in the nucleus of mononuclear cells from fetal blood with a single X chromosome or monosomy X (Turners syndrome).

FIG. 7 shows cells from chorionic villus, amniotic fluid and fetal blood.

FIG. 7A(i) shows CEP X and CEP Y, and 7A(ii) shows normal diploid in chromosome 21, in nucleus of trophoblast cells from chorionic villus sample.

FIG. 7B(i) shows CEP X, and 7B(ii) shows normal diploid in chromosome 21, in nucleus of uncultured amniocytes.

FIG. 7C(i) shows CEP X and CEP Y, and 7C(ii) shows trisomy in chromosome 21, in nucleus of mononuclear cells from fetal blood sample.

### DEFINITIONS

Biopolymer—Any molecule such as nucleic acids or proteins from living tissue. In this application, the term “polypeptide” and “protein” are used interchangeably.

Fixing—In histology, fixing is the preservation and hardening of a cell or tissue sample to retain as nearly as possible the same relationship the cellular or tissue components had in the living body. Chemicals used for fixing are called fixatives.

Complementary—Generally, nucleic acid consists of nitrogenous bases that are either pyrimidines (Cytosine (C), uracil (U), and thymine (T) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” in nucleic acids refers to the base pairing that occurs between two distinct nucleic acid sequences or two distinct regions of the same nucleic acid sequence. Proteins consist of three dimensional arrangements of amino acids. “Complementary” in proteins refer to the fitting and binding of molecules (typically other proteins) to proteins.

Accordingly, nucleic acids and proteins can have complementary molecules that bind or hybridise to them and such molecules can be used to detect the biopolymers that they are complementary to. It is usual in the art to refer to the biopolymers to be detected as “targets” and the complementary molecules as “probes”.

Binding and/or Hybridisation—The binding and/or hybridisation of molecules to the nucleic acid and/or polypeptide is carried out by incubating targets and the probes for certain time periods and at certain temperatures to allow the molecules to bind. Detection of molecules can be by the use of a detactable label molecule attached to the complementary molecule.

While the term “hybridisation” may be commonly used in the art to refer to binding of complementary nucleic acids, for the purposes of the present invention the term “hybridisation” and “binding” will be used to indicate in general, the binding of molecules to nucleic acid(s) and/or polypeptide(s).

Accordingly, for the purpose of the present invention, the “binding” and/or “hybridisation” of molecule(s) to nucleic acid(s) will not require the complete match (complementarity) of any single nucleotide of the binding molecule (probe) to the target nucleic acid. As such, the term “in situ hybridisation” also refers to the detection, visualization and location of nucleic acids and/or polypeptides under the present invention.

Protein digestion—the complete or partial breakdown of a protein molecule, typically by an acid or an enzyme (a protease). Partial digestion can change or cause a loss of the three-dimensional shape of the protein while complete digestion can reduce a protein into its component amino acid molecules.

Denaturation—Reduction in the three-dimensional shape of a biopolymer such as a nucleic acid or protein. The denaturation may be partial or complete. In nucleic acids, it refers to the separation of two complementary strands of nucleic acids. In proteins, it refers to a change in the three-dimensional shape of the protein.

### DETAILED DESCRIPTION OF THE INVENTION

Bibliographic references mentioned in the present specification are for convenience listed in the form of a list of references and added at the end of the example. The whole content of such bibliographic references is herein incorporated by reference.

A person skilled in the art will appreciate that the present invention may be practised without undue experimentation according to the method given herein. The methods, techniques and chemicals are as described in the references given or from protocols in standard biotechnology and molecular biology text books. Standard molecular biology techniques known in the art and not specifically described were generally followed as described in Sambrook and Russel, Molecular Cloning: A Laboratory Manual, Cold Springs Harbor Laboratory, New York (2001).

While the method of the present invention is optimised for the detection of biopolymers such as nucleic acids, a person skilled in the art will recognise that it may also be readily adapted for the detection of polypeptides or proteins as the digestion of polypeptides is not performed. In particular, the present invention does not require the use of protease to digest polypeptides.

A person skilled in the art will appreciate that an in situ hybridisation method such as the method of the present invention detects the presence or absence of one or more biopolymers of interest in a sample. The presence or absence or one or more biopolymers of interest may then be used to indicate any abnormalities within the sample or the subject from which the sample was drawn.

The method according to the invention may be used to detect the presence or absence of certain nucleic acids or polynucleotides. As FISH may be used on amniotic cells drawn from pregnant female subjects, the absence, presence or number of these polynucleotides can indicate whether chromosomal abnormalities are present. For example, should a probe directed towards a region of chromosome 21 detect three copies of the chromosome in each nucleus for a statistically significant number of cells in the sample, the result indicates the condition of Trisomy 21 wherein the chromosomal abnormality of an additional copy of chromosome 21 is seen. Similarly, should only one copy of the X chromosome be detectable in a sample from another subject, the chromosomal abnormality or the genetic condition of Monsomy X is indicated. Besides the absence (deletion) of a chromosome, the presence of an additional chromosome (duplication), FISH may also be used to highlight other chromosomal abnormalities such as ring formation, inversions, translocations and other unnatural conformations of chromosomes in chromosomes of interest. Accordingly, with the term “chromosomal abnormality” it is understood the presence of any nucleic acid or chromosomal difference compared to the normal nucleic acid or chromosomal status. Therefore “chromosomal abnormality” included, but it is not limited to: any variation of number of chromosomes, trisomy, monsomy, complete absence of chromosome, deletion, inversion, translocation, ring formation, or any other unnatural conformation of chromosomes. Thus, the present invention of rapid FISH will be referred to as FastFISH in the subsequent sections of the specification. This method may be used to detect the absence or presence of a particular polynucleotide or to quantify the presence of it, and there from, to detect and/or identify any chromosomal abnormalities present.

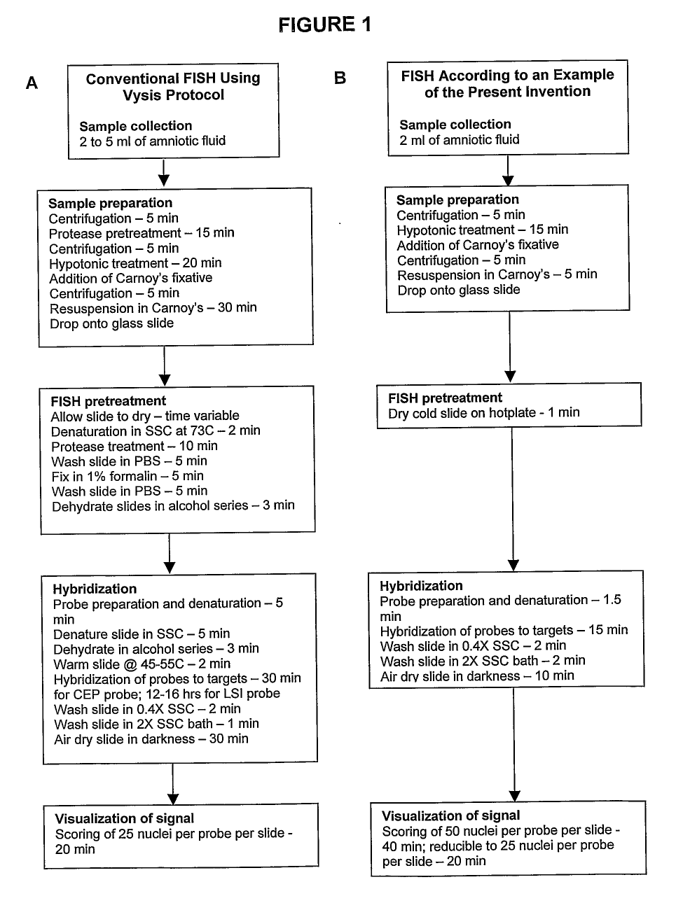

To describe the present invention, reference will be made to the standard protocol for the commercial Centromeric Enumeration Probe (CEP) and Locus-Specific (LSI) Probe supplied by the biotechnology company Vysis (Downers Grove, Ill., USA). Both CEP and LSI are registered trademarks of Vysis. The Vysis FISH protocol for use of the CEP and LSI probes, as given in their website (http://www.vysis.com/VysisProducts_{—}32945.asp) with a treatment protocol for amniotic specimens (http://www.vysis.com/Specimen_{—}32946.asp) are summarised in FIG. 1A.

The present invention is an improvement to existing protocols of FISH, allowing the procedure to be completed in a shorter time than the FISH protocols of the prior art. In particular, the present invention may be performed in less than two hours for the CEP and LSI probes supplied by Vysis. Under the method of the present invention, the sample only needs a hybridisation time of 30 min for both probes. This shorter time period is contrasted with that of the Vysis protocol that requires hybridisation periods of 30 min for the CEP probe and 12-16 hours for the LSI probe.

### METHOD OF THE PRIOR ART

### Sample Preparation of the Prior Art

The Vysis FISH protocol using the CEP and LSI probes, as given in their website (http://www.vysis.com/VysisProducts_{—}32945.asp) and a treatment protocol for amniotic specimens (http://www.vysis.com/Specimen_{—}32946.asp) are summarised in FIG. 1A as well as described as follows.

The method of the prior art is summarized in FIG. 1A. Two to five mililiters (ml) of clear amniotic fluid is centrifuged for 5 min to pellet the amniotic cells. The cells are then resuspended in a buffer with the protease trypsin at 37° C. for at least 15 min to reduce the amount of protein present and to make the cells more permeable to the probe. The trypsin is then removed by centrifuging the cells for 5 min and resuspending the cells in 2-5 ml of a hypotonic (0.56% KCI) solution, followed by incubation of 20 minutes in a 37° C. water bath to swell the cells.

The cells are then fixed with 0.8-2.0 ml of Carnoy's fixative (3:1 methanol: glacial acetic acid). The suspension is then centrifuged for 5 min and the pellet resuspended in 1 ml of fresh Carnoy's fixative. This suspension is then to be kept at 4° C. for at least 30 minutes before performing pretreatment step for FISH.

### Pre-Treatment of Sample of the Prior Art

Cells are dropped onto cold glass slides (15-25 μL of cell suspension per drop) and allowed to completely dry at room temperature to fix the cells onto the slides. This drying time may take up to 30 min, depending on the ambient humidity. The slides are then immersed in 2×SSC (sodium chloride and sodium citrate buffer) for 2 min at 73° C. to denature duplex nucleic acids. The slides are then immersed in a second protease solution of pepsin for 10 min at 37° C. to reduce the proteins that are usually associated with the nucleic acids.

The slides are then washed in 1×PBS (phosphate buffered saline) for 5 min at room temperature before being fixed in 1% formaldehyde for 5 min at room temperature. The slides are then washed in 1×PBS for 5 min at room temperature and dehydrated in an alcohol series (1 min each in 70%, 85% and 100% ethanol) to ‘age’ the nucleic acids, so as to preserve their shape.

### Hybridisation Protocol of the Prior Art

Probe (either CEP or LSI; 1 μL) is mixed with 7 μL of buffer and 2 μL of water are mixed and heated for 5 min at 73° C. in a water bath to denature the probes in case there are any self-complementary hybridisations within the probe sequence. The probes are then maintained at 45-50° C. on a slide warmer. The slides with the cell samples are immersed into a denaturant bath of 70% formamide and 2×SSC at 73° C. for 5 min and then dehydrated in an alcohol series (1 min each in 70%, 85% and 100% ethanol). The slides are then dried and placed on a slide warmer at 45° C. to 55° C. for up to 2 min.

For hybridisation, 10 μL of probe mix is placed on each area of the slide containing the cell sample and allowed to hybridize. For CEP probe, the hybridisation temperature is 42° C. and the duration is between 30 min and 60 min. For LSI probe, the hybridisation temperature is 37° C. and the duration is between 12 hr and 16 hr.

After hybridisation, the slides are washed in a solution of 0.4×SSC and 0.3% NP-40, a non-ionic surfactant, for 2 min at 73° C., followed by another wash in a 2×SSC and 0.1% NP-40 solution for up to 1 min. The slides are then air-dried in darkness; this may take up to 30 minutes, depending on the ambient humidity. The probes are then visualized by epifluorescence microscopy.

It will be appreciated by one skilled in the art that the above protocol of the prior art takes about 3.2 hours to complete for the CEP probe. Time for the LSI probe is significantly longer because of the recommended 12-16 hrs of hybridisation time. It will also appear to a person skilled in the art that all the above steps are essential or required to render the biopolymer (e.g. nucleic acids in this case) in the sample amenable to hybridisation with the probe (or complementary molecule that is capable of being detected or visualized).

These steps include those for protease treatments, hypotonic solution treatment, fixative treatment, denaturation, maintenance of hybridisation temperatures and duration, washing to remove unbound probe. No suggestion or indication is given in the art that any of these steps can be eliminated to shorten the time needed to obtain the results of amniocentesis so as to reduce anxiety of the expectant mother. More in particular, it will not be apparent to a skilled person to remove the protease treatment(s) step which is considered to be essential in the method of the prior art.

### METHOD OF THE PRESENT INVENTION

The present invention provides an improved and/or efficient method of in situ hybridisation.

According to a first aspect, the present invention provides a method for in situ hybridisation of nucleic acids and/or polypeptides in at least one cell. A sample of cells is used in the example below to detect two nucleic acids. In particular, the invention provides a method of in situ hybridisation with the proviso that the digestion of polypeptides is not performed.

Accordingly, the present invention provides a method for in situ hybridisation of nucleic acid(s) and/or polypeptide(s) in at least one isolated cell with the proviso that the method does not comprise digestion of polypeptide(s). There is also provided a method for in situ hybridisation of nucleic acid(s) in at least one isolated cell with the proviso that the method does not comprise digestion of polypeptide(s).

In particular, the present invention provides a method for in situ hybridisation of nucleic acids and/or polypeptides in at least one isolated cell comprising the steps of: providing at least one isolated cell; contacting the at least one cell with at least one hypotonic solution; treating the at least one cell with at least one fixative; providing at least one molecule capable of binding to at least one nucleic acid and/or polypeptide, and allowing binding of the at least one molecule to the nucleic acid(s) and/or polypeptide(s) for a time period; removing any unbound molecule(s); and detecting any bound molecule(s), with the proviso that method does not comprise digestion of polypeptides. Under the method, the at least one isolated cell is applied to a surface after treatment with the at least one fixative. The nucleic acids and polypeptides may be subjected to a denaturing step before the binding and the denaturing step may be performed by heat.

The method may be used for detection of nucleic acids and polypeptides in the same sample of cells by use of at least one (first) molecule capable of binding or hybridising to a nucleic acid and/or at least a (second) molecule capable of binding or hybridising to a polypeptide.

The at least one molecule capable of binding or hybridising to at least one nucleic acid and/or polypeptide may be at least one molecule complementary to the at least one nucleic acid and/or polypeptide.

Further, the molecule(s) capable of binding to the nucleic acid(s) and/or polypeptide(s) may be labeled with at least one label selected from the group consisting of fluorescer, chemiluminescer, enzyme label, radiolabel and a mixture thereof.

Under the method of the present invention, the step comprising binding may comprise at least one first temperature exposure at a temperature range from 70° C. to 90° C. for 60 sec to 120 sec. In particular, the first temperature exposure may be at a temperature range from 75° C. to 85° C. for 80 sec to 100 sec. More in particular, the first temperature exposure is a temperature of approximately 80° C. for 90 sec. This step also comprises at least one subsequent temperature exposure at a temperature range from 37° C. to 47° C. In particular, the at least one subsequent temperature exposure is performed at a temperature range from 40° C. to 45° C. More in particular, the subsequent temperature exposure is performed at a temperature of approximately 42° C.

The present inventor found that the elimination of polypeptide digestion step(s), for example avoiding using pepsin and/or trypsin, not only save time, but also reduced excessive spreading of the nuclei. For example, it will be appreciated that by obviating the first protease treatment for sample preparation under the present invention, may save about 20 min over the prior art.

Further, the person skilled in the art will appreciate that for the hybridisation step of the present invention, the hybridisation time for the CEP probe may be shortened from at least 30 min to 15 min. The reduction for the LSI probe may be even more dramatic, for example, from at least 12 hours to a mere 15 min, with no compromise or substantial compromise in the quality of the signal.

The cells are eukaryotic, for example mammalian cells. The mammalian cells may be human cells or non-human cells. More particularly, the cells may be human amniotic cells. According to one aspect of the invention, the method may be used for detecting chromosomal abnormalities in amniotic cells. In particular, the amniotic cells or amniocytes may be obtained from pregnant mammals. Also, while amniocytes are mainly used in this study and other cell types had been tested and shown to have successful hybridisation. Therefore, other cell types for example lymphocytes, fetal nucleated red blood cells, white blood cells, mononuclear cells, cancer cells, trophoblast cells, and the like, can be used for this protocol using different fluorescence probes.

The person skilled in the art will also appreciate that the obviation of protease treatment(s) may also allow the use of protein probes to detect polypeptides of interest, something that is not possible in the prior art. Accordingly, in the method of the invention, the method may also be used for detecting presence of at least one polypeptide in a sample of cells. While the present invention has been demonstrated for detection of DNA as the biopolymer of interest, it will be apparent to a person skilled in the art that the present invention may also be applied to other biopolymers such as RNA and/or polypeptides. The method of visualization of the results can be readily extended to use of other labels such as enzyme labels and/or radiolabels.

It can be further appreciated by one skilled in the art that because protease treatment is obviated, the detection of the two kinds of biopolymers cited (nucleic acids and polypeptides) to the same sample of cells can now be performed, under the method of the present invention. For example, a first kind of molecule(s) or probe(s) which binds, hybridises or is complementary to a first kind of biopolymer target such as nucleic acid(s), may be labelled with at least a first kind of label, and a second kind of molecule or probe which binds, hybridises or is complementary to a second kind of biopolymer target such as protein(s) may be labelled with at least a second kind of label. Suitable types of labels may be fluorescers, chemiluminescers, enzyme labels and radiolabels.

After hydridization to their respective targets in the same sample, these probes may then be visualized. If the labels for the two kinds of probes are the same (eg fluorescers), then they may be visualized together in the same image. If the labels are of different kinds, they may be separately visualized and the separate images obtained may be superimposed, if desired, to compare localization of both labels.

There is also provided a kit for assaying the presence of a suspect nucleic acid and/or polypeptide in a cell sample. The kit comprises at least one hybridisation buffer salt, a denaturing agent, a hybridising agent, a salt for a hypotonic solution, a fixative, at least one molecule capable of binding to at least one nucleic acid and/or polypeptide and capable of being detected. The kit, optionally, may further comprise information pertaining to the kit, for example, instruction for the use of the kit and/or the individual components. In particular, the kit does not comprise any agent or means for performing the digestion of the polypeptides. More in particular, the kit does not comprise any protease. More in particular, there is provided a kit wherein the kit is for detecting chromosomal abnormalities is amniotic cell(s). A person skilled in the art will also appreciate that alternative chemicals or reagents can be substituted to achieve the same function, for example, instead of PBS, a different buffer may be used.

Having now generally described the invention, the same will be more readily understood through reference to the following example that is provided by way of illustration, and are not intended to be limiting of the present invention.

### EXAMPLE

To demonstrate the robustness of the method of the present invention, namely FastFISH, the following clinical study was performed on 100 uncultured amniotic fluid samples. All amniotic fluid samples were clear, with no macroscopic evidence of blood-cell contamination. The indications for amniocentesis were maternal age ≧35, positive screening test for Downs syndrome (nuchal translucency or maternal serum markers) or abnormal fetal ultrasound scan. One chorion villus sample, one amniotic fluid and one fetal blood sample was also collected for one day validation study. The median gestational age was 16 weeks (14-24 weeks), and the maximum time taken to analyse each of the 100 samples was 2 hours.

Prior to the clinical study, the technique was tested on samples of uncultured amniotic fluid (AF) cells (n=18), chorionic villus (CVS) biopsy cells (n=5), fetal nucleated blood cells, umbilical cord nucleated blood cells and nucleated cells from neonatal blood (n=8), in known pregnancies and the pregnancies complicated by triosomies 13, 18 and 21. FastFISH was tested for Turner syndrome using adult peripheral blood samples. After density gradient centrifugation, nucleated blood cells were processed in the same way as uncultured amniotic fluid cells, obviating several days of culture, metaphase preparation, overnight aging and prolonged hybridisation.

In contrast to the long procedure of the prior art, the FastFISH method of the present invention will now be described with reference to FIG. 1B. It will be clear to a skilled person that the following example refers to a particular protocol, however the method of the present invention should not be construed to be limited to the particulars steps, chemicals, reagents, times, and the like.

### Sample Preparation of the Present Invention

A 2 ml sample of amniotic fluid comprising amniotic cells drawn from 100 pregnant females was collected in the same way as described in the prior art. The sample of cells was centrifuged for 5 min at 1800 rpm and the cell pellet was suspended in 3 ml of pre-warmed 0.075M KCI hypotonic solution for 30 min at 37° C. The cells were then fixed by the drop-wise addition of 2 ml of Carnoy's fixative and then centrifuged for 5 min at 1800 rpm. The cell pellet was then resuspended in Carnoy's fixative for 5 min. The cells were then applied by drops onto the surfaces of cold slides and placed on a 60° C. hot plate for 1 min to affix the cells onto the slides and to simultaneously denature the biopolymers of interest. These slides may then be subjected to the hybridisation step.

It can be seen that the first protease treatment and centrifugation steps have been obviated for sample preparation under FastFISH method of the present invention, saving 20 min over the prior art. Also obviated were the laborious pre-treatment steps of denaturation in SSC buffer at 73° C., the second protease treatment, slide washes, fixation in 1% formalin, another wash, and dehydration in an alcohol series. Omission of these pre-treatment steps results in an additional reduction of up to 59 minutes over the prior art.

### Hybridisation Protocol of the Present Invention

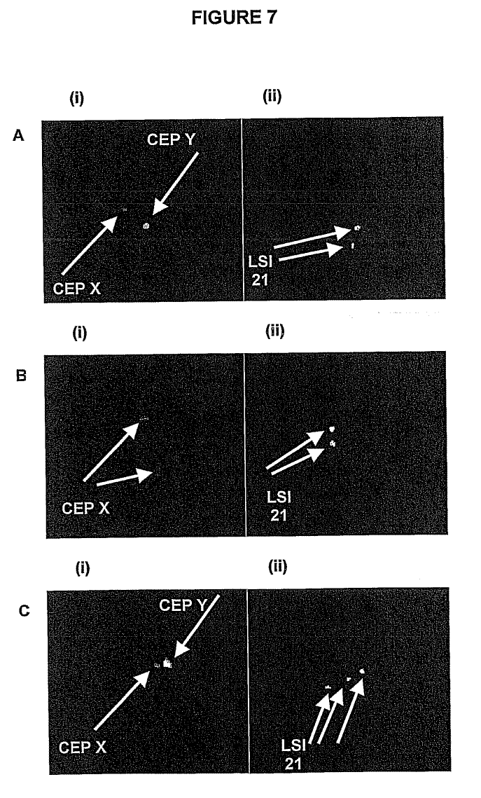

For each of the sample, two slides were prepared. On one of the slides 2 μL of Centromeric Enumeration Probe (CEP) X/Y probe (Vysis Inc, Downers Grove, Ill., USA) mixed with 3 μL of the hybridisation buffer (50% formamide and 10% dextran sulphate in 2×SSC, pH 7.0) was added to each cell spot of sample on the slide. The cell spots on the second slide was hybridised with locus-specific (LSI) 21 probe (Vysis) in the presence of hybridisation buffer as described above. The slides were covered with a coverglass and sealed with Parafilm (American National Can Company, Chicago, Ill., USA). A representative result of hybridisation using these probes on two different slides is shown in FIG. 7B.

Target DNA on the slides were then denatured on an in situ hybridisation heating block (MJ Research PTC-200, Waltham, USA) at 80° C. for 90 sec followed by hybridisation for 15 min at 42° C. for both probes. The slides with the CEP probe were then washed with agitation in 0.4×SSC/0.3% NP-40 at 72° C. for 2 min and in 2×SSC/0.1% NP-40 at room temperature for 2 min. Slides with the LSI probe gave better signals when washes were, for 1 min each with less agitation.

The slides were allowed to dry in the dark. Optionally, the drying the slides may be done in the presence of a desiccant such as silica gel or under a gentle stream of dry nitrogen which speeds up the drying time significantly from 20 min to less than 10 min. The slides were then mounted in fluorescence antifade medium containing DAPI (Vector Laboratories, Burlingame, Calif., USA) and analysed in epifluorescence microscope fitted with Xenon lamp (Olympus BX61, Center Valley, USA).

When establishing the FastFISH protocol of the present invention, 50 nuclei per slide were scored without image enhancement for each probe, taking up to 40 min per slide. A sample considered is euploid or aneuploid if at least 80% of the nuclei displayed respectively the same normal or abnormal hybridisation pattern for any specific probe. Image capture was performed using FISHView, 2.0 EXPO (ASI, Carlsbad, USA) and SPSS 14.0 (SPSS Inc, Chicago, Ill.) was used for the statistical analysis. However, owing to the robustness of the method of the present invention (see below), as few as 25 nuclei can be scored in the future, hence halving the time required for this step.

In this blinded study, FastFISH was performed for chromosomes X, Y and 21, to verify that the technique was suitable for both CEP and LSI commercial probes. FastFISH was also performed for chromosomes 13 and 18 where multiple fetal malformations were present on ultrasound scan, and for the 22q deletion where conotruncal abnormalities were noted. These were in addition to FastFISH for chromosomes 21, X and Y. Of the 100 consecutive participants, a conotruncal abnormality was found in one fetus during ultrasound. FastFISH was performed for 22q11.2 deletion to exclude DGS; this was in addition to FastFISH for chromosomes 21, X, Y, 13 and 18. In this blinded study, conventional karyotype results were disclosed to the investigators performing FastFISH only after all 100 amniotic fluid cases had been analysed and recorded, and the FastFISH results were not disclosed to the cytogeneticists performing the karyotype.

For the validation of one day testing, FastFISH for chromosomes 21, X and Y was performed on CVS, AF and fetal Blood samples (11, 15, 23 gestational weeks respectively) processed in the same way as described for amniotic fluid. All the steps were completed on the same day of sample collection.

### RESULTS

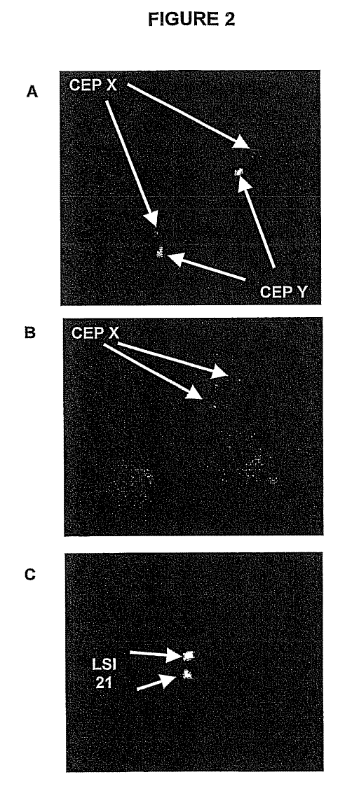

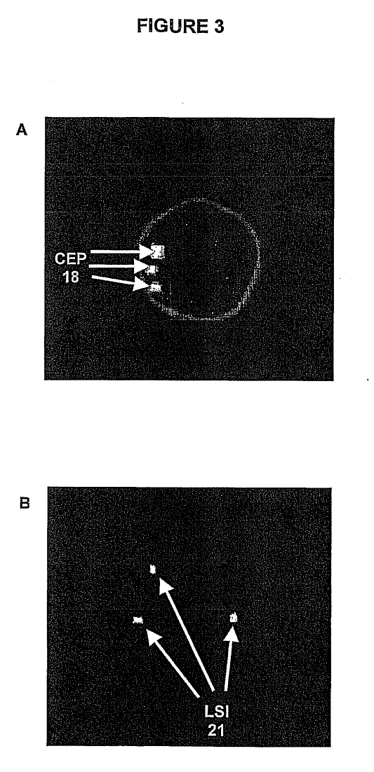

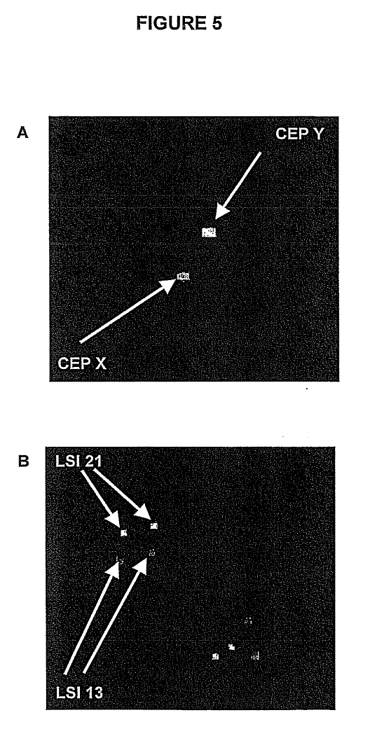

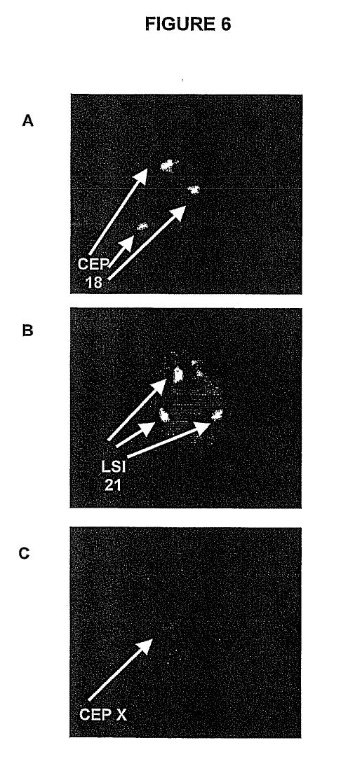

Representative results on uncultured amniotic cells are shown in FIGS. 2 to 4 and results obtained on fetal, cord and neonatal blood are shown in FIGS. 5 and 6. The validation of one day testing using FastFISH for chromosomes 21, X and Y was performed on CVS, AF and fetal Blood samples is shown in FIG. 7.

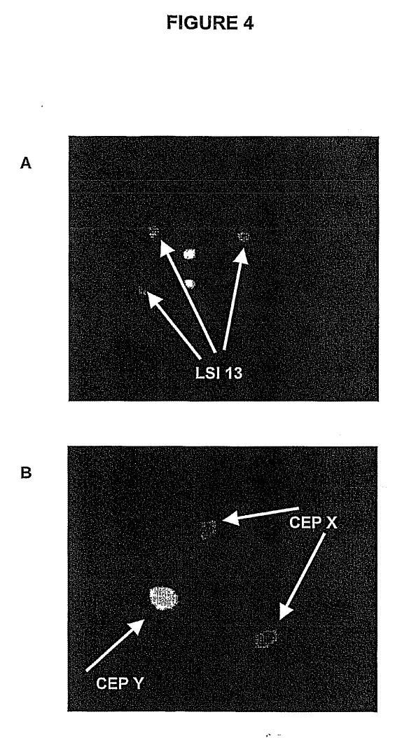

In this blinded clinical study, all amniotic fluid samples were free of macroscopic blood contamination. Median maternal age was 36 years (range 24-45), the median gestational age was 16 weeks (14-24), and the maximum time taken to analyse each of the 100 samples was 2 hours. There were 49 male (XY), 50 female (XX) fetuses and one 47, XXY (Klinefelter syndrome) fetus. All these cases were identified correctly by the FastFISH method of the present invention as described in the example in the preceeding section. For the fetus with Klinefelter syndrome 100% of nuclei demonstrated the sex chromosome trisomy (FIG. 4B). In three cases, at least 80% of nuclei had three signals for the chromosome 21 probe (80.0%, 84.4% and 83.3%); all three fetuses were confirmed by conventional karyotyping to have trisomy 21. More than 50 nuclei were available for evaluation in the first of these cases and 80.0% of the 50 evaluated nuclei showed trisomy 21. In the second case, only 45 nuclei could be evaluated and 38 of them showed trisomy 21 (84.4%), while in the third case 30 of 38 (83.3%) nuclei evaluated showed trisomy 21; in all other 97 cases, more than 50 nuclei were available for evaluation of trisomy condition. Examination of 50 nuclei from the rest of these 97 fetuses revealed that they were disomic for chromosome 21. One case with conotruncal abnormalities detected on ultrasound at time of amniocentesis was evaluated for DGS; the FastFISH method of the present invention revealed no deletion of 22q11.2. The FastFISH for chromosomes 21, X, Y, 13 and 18 also revealed aneuploidies. These results were later confirmed by conventional FISH and karyotyping respectively.

In all cases gender, trisomy and chromosomal rearrangement (deletion) status were identified correctly (100% accuracy; lower 95% CI, 97.05%). In this, blinded study the sensitivity was 100%, specificity was 100% (three cases of trisomy 21 and one case of Klinefelter syndrome) and there were no false-positive or false-negative results. Hybridisation was satisfactory in all cases, and there were no uninformative cases. The time taken from sample collection to release of FastFISH results for 57 samples was evaluated and representative recording for 1 to 5 samples per day is shown in Table 1. All the cases were processed by the same technician. One technician can readily complete up to at least 2 samples (6 hours) on the same day. A maximum of 4 samples to 5 samples in 10 to 12.5 hours respectively can be completed by the end of the same day.

TABLE I
Total time taken from collection to release of results as analysed in 57
samples.

| No. of | Total time from collection to | |
|---|---|---|
| samples/day | release of results (hours to | |
| (2 slides/ | complete all samples; 2 slides per | No. of days |
| sample) | sample; 21/XY) | (times) tested |
| 1 | 4 | 1 |
| 2 | 6 | 3 |
| 3 | 7.5 | 2 |
| 4 | 10 | 6 |
| 5 | 12.5 | 4 |

For the validation of the one day testing, the FastFISH method of the present invention for chromosomes 21, X and Y was performed on a sample of CVS, AF and fetal blood (11, 15, 23 gestational weeks respectively) within the same day of sample collection and results were released on the same day. The chorionic villus and amniotic fluid samples were diagnosed as disomic for chromosome 21 whereas the fetal blood sample revealed trisomy 21 (FIG. 7). All the blinded study and one-day testing results matched the conventional testing methods.

The person skilled in the art will appreciate that for the hybridisation step of the present invention, the hybridisation time for the CEP probe has been shortened from at least 30 min to 15 min. The reduction for the LSI probe is even more dramatic, from at least 12 hours to a mere 15 min, with no compromise in the quality of the signal.

Several inventive changes to the conventional protocol of the prior art have been implemented. Usually, up to 5 ml of amniotic fluid are collected for FISH, possibly to save some as backup in case of failed hybridisation. It was found that 2 ml of amniotic fluid were adequate in all cases. Also steps as washing, sonicating and/or coating glass slides could be obviated; using clean slides was sufficient for good hybridisation and signal quality.

Elimination of protein digestion steps using pepsin or trypsin not only saved time, but also reduced excessive spreading of the nuclei. By warming the cold slide on hot plate, close proximity of the nuclei (clustering) without causing clumping or overlapping signals was ensured. Even without ethanol dehydration, most FISH signals could be readily visualised on the same focal plane. Any value in the aging of slides was doubtful and this step was deleted. Finally, 15 minutes was sufficient time for hybridisation of both CEP and LSI probes. Incorporating these changes, and allowing some time for logistics purposes such as moving between laboratories to use specialised equipment, FISH on uncultured amniotic fluid cells could be performed within two hours in each case, compared to the 3.3 hrs for the method of the prior art. Use of automated image analysers could further reduce the labour demand of the technique, and make it even faster.

As a whole, these combination of steps greatly extends beyond mere workshop improvements and renders the method inventive as underscored by the surprising result that the hybridisation time needed for the LSI probe is no longer 12-16 hr but a mere 15 min. In addition to the savings in time, the amount of materials required are also reduced, making the method of the present invention more economically attractive to perform compared to conventional FISH protocols.

### REFERENCES

Bryndorf T, Christensen B, Vad M, Parner J, Brocks V, Philip J. Prenatal detection of chromosome aneuploidies by fluorescence in situ hybridization: experience with 2000 uncultured amniotic fluid samples in a prospective preclinical trial. Prenat Diagn 1997; 17(4):333-41.
Caccia N, Johnson J M, Robinson G E, Barna T. Impact of prenatal testing on maternal-fetal bonding: chorionic villus sampling versus amniocentesis. Am J Obstet Gynecol 1991; 165(4 Pt 1):1122-5.
Evans M I, Bottoms S F, Carlucci T, et al. Determinants of altered anxiety after abnormal maternal serum alpha-fetoprotein screening. Am J Obstet Gynecol 1988; 159(6):1501-4.
Jouannic J M, Martinovic J, Bessieres B, Romana S, Bonnet D. Fluorescence in situ hybridization (FISH) rather than ultrasound for the evaluation of fetuses at risk for 22q11.2 deletion. Prenat Diagn 2003; 23(7):607-8. Klinger K, Landes G, Shook D, et al. Rapid detection of chromosome aneuploidies in uncultured amniocytes by using fluorescence in situ hybridization (FISH). Am J Hum Genet 1992; 51(1):55-65.
Marteau T M, Cook R, Kidd J, et al. The psychological effects of false-positive results in prenatal screening for fetal abnormality: a prospective study. Prenat Diagn 1992; 12(3):205-14.
Pertl B, Yau S C, Sherlock J, Davies A F, Mathew C G, Adinolfi M. Rapid molecular method for prenatal detection of Down's syndrome. Lancet 1994; 343(8907):1197-8.
Ward B E, Gersen S L, Carelli M P, et al. Rapid prenatal diagnosis of chromosomal aneuploidies by fluorescence in situ hybridization: clinical experience with 4,500 specimens. Am J Hum Genet 1993; 52(5):854-65.

Witters I, Devriendt K, Legius E, at al. Rapid prenatal diagnosis of trisomy 21 in 5049 consecutive uncultured amniotic fluid samples by fluorescence in situ hybridisation (FISH). Prenat Diagn 2002; 22(1):29-33.

1. A method for in situ hybridisation of nucleic acids and/or polypeptides in at least one isolated cell comprising the steps of:
(a) providing at least one isolated cell;
(b) contacting the at least one cell with at least one hypotonic solution;
(c) treating the at least one cell with at least one fixative;
(d) providing at least one molecule capable of binding to at least one nucleic acid and/or polypeptide, and allowing binding of the at least one molecule to the nucleic acid(s) and/or polypeptide(s) for a time period;
(e) removing any unbound molecule(s); and
(f) detecting any bound molecule(s),
with the proviso that the method does not comprise digestion of polypeptides.

2. The method according to claim 1, wherein at least one molecule comprises at least one molecule complementary to at least one nucleic acid or to at least one polypeptide.

3. The method according to claim 1 or claim 2, wherein the at least one isolated cell is applied to a surface after step (c).

4. The method according to any one of claims 1 to 3, wherein the nucleic acid(s) and/or polypeptide(s) are subjected to a denaturing step before step (d).

5. The method according to claim 4, wherein the denaturing step is performed with heat.

6. The method according to claim 5, wherein the denaturing step is performed at a temperature range of 50° C. to 70° C.

7. The method according to any one of claims 5 to 6, wherein the denaturing step is performed at a temperature range of approximately 60° C.

8. The method according to any one of claims 1 to 7, wherein the at least one molecule capable of binding to at least one nucleic acid and/or polypeptide is labeled with at least one label selected from the group consisting of fluorescer, chemiluminescer, enzyme label and radiolabel.

9. The method according to any one of claims 1 to 8, wherein the binding time period of step (d) is less than 60 minutes.

10. The method according to claim 9, wherein the binding time period is less than 45 minutes.

11. The method according to claim 9, wherein the binding time period is less than 20 minutes.

12. The method according to any one of claims 1 to 11, wherein step (d) comprises at least one first temperature exposure at a temperature range from 70° C. to 90° C. for 60 sec to 120 sec.

13. The method according to claim 12, wherein the first temperature exposure is at a temperature is approximately 80° C. for 90 sec.

14. The method according to any one of claims 12 to 13, wherein step (d) further comprises at least one subsequent temperature exposure at a temperature range from 37° C. to 47° C.

15. The method according to claim 14, wherein the at least one subsequent temperature exposure is performed at a temperature of approximately 42° C.

16. The method according to any one of claims 1 to 15, wherein the at least one cell is not dehydrated.

17. The method according to any one of claims 1 to 16, wherein the at least one cell is a mammalian cell.

18. The method according to claim 17, wherein the at least one cell is a human cell.

19. The method according to any one of claims 17 to 18, wherein at least one cell is an amniotic cell.

20. The method according to any one of claims 1 to 19, wherein the method is for detecting chromosomal abnormalities in amniotic cells.

21. The method according to any one of claims 1 to 20 wherein the method is for detecting presence of at least one polypeptide in a sample of cells.

22. The method according to claim 20, wherein the cells are obtained from pregnant mammals.

23. The method according to any one of claims 1 to 22, wherein the method further comprises a step of diagnosis.

24. The method according to claim 23, wherein the step of diagnosis comprises determining the presence or absence of chromosomal abnormalities in the cell(s) of a mammal.

25. The method according to any one of claims 1 to 24, wherein the method is for determining the normality or abnormality of a fetus.

26. A kit for assaying the presence of nucleic acids and/or polypeptides in a cell sample comprising at least one denaturing agent, at least one hybridising agent, at least one salt for a hypotonic solution, at least one fixative, and at least one molecule capable of binding to at least one nucleic acid or polypeptide and capable of being detected.

27. The kit according to claim 26, wherein the kit does not comprise any agent or means for performing the digestion of the polypeptides.

28. The kit according to any one of claims 26 to 27, wherein the kit does not comprise any protease.

29. The kit according to any one of claims 26 to 28, wherein the kit is for detecting chromosomal abnormalities is amniotic cell(s).

30. The kit according to any one of claims 26 to 29, wherein the kit is for determining the normality or abnormality of a fetus.

31. A method for detecting chromosomal abnormalities in amniotic cells comprising the steps of:
(a) providing at least one cell isolated from amniotic fluid;
(b) contacting the at least one cell with at least one hypotonic solution;
(c) treating the at least one cell with at least one fixative;
(d) providing at least one molecule capable of binding to at least one nucleic acid and/or polypeptide, and allowing binding of the at least one molecule to the nucleic acid(s) and/or polypeptide(s) for a time period;
(e) removing any unbound molecule(s);
(f) detecting any bound molecule(s): and
(g) detecting the presence or absence of chromosomal abnormalities, with the proviso that the method does not comprise digestion of polypeptides.

32. The method according to claim 31, wherein the presence of chromosomal abnormality(ies) correlates to the abnormality of a fetus.
